(19) United States
(12) Patent Application Publication
(10) Pub. No.: US 2024/0055105 A1
(43) Pub. Date: Feb. 15, 2024

(54) REAL-TIME ANALYSIS OF IMAGES CAPTURED BY AN ULTRASOUND PROBE

(71) Applicant: Echo Mind AI Corp, Highlands Ranch, CO (US)

(72) Inventors: Leo Max Harker, Highlands Ranch, CO (US); Darren S. Lund, Provo, UT (US); Casey Kiane Charlebois, Clinton Township, MI (US); Manuel Rodrigo Parra Castaneda, Chicago, IL (US)

(21) Appl. No.: 18/448,671

(22) Filed: Aug. 11, 2023

Related U.S. Application Data
(60) Provisional application No. 63/397,594, filed on Aug. 12, 2022.

(51) Int. Cl. G16H 30/40; G06T 7/00; G16H 15/00; G16H 30/20; G16H 50/20

(52) U.S. Cl. CPC G16H 30/40; G06T 7/0012; G16H 15/00; G16H 30/20; G16H 50/20; G06T 2207/10132

(57) ABSTRACT

The present application describes a musculoskeletal diagnosis system that receives, in real-time or substantially real-time, various images from a musculoskeletal ultrasound probe. Once the images are received, the musculoskeletal diagnosis system analyzes the various images to detect various pathologies.

### CROSS-REFERENCE TO RELATED APPLICATIONS

The present application claims priority to U.S. Provisional Application 63/397,594 entitled “REAL-TIME ANALYSIS OF IMAGES CAPTURED BY AN ULTRASOUND PROBE”, filed Aug. 12, 2022, the entire disclosure of which is hereby incorporated by reference in its entirety.

### BACKGROUND

Ultrasound images may be used to enable a doctor to identify different musculoskeletal conditions (e.g., tears and strains). However, doctors are typically not trained to capture these images. As such, if a doctor determines that an individual needs an ultrasound, the doctor refers the individual to an ultrasound technician. Once the individual is referred to the ultrasound technician, the individual must schedule an appointment with the ultrasound technician and subsequently schedule a follow-up appointment with the doctor. This process can take a number of weeks.

### SUMMARY

The present application describes a musculoskeletal diagnosis system that receives, in real-time or substantially real-time, various images from a musculoskeletal ultrasound probe and analyzes the various images to detect various pathologies. The musculoskeletal diagnosis system may generate a report using selected images and also generate information for the report. The information may include a diagnosis and/or one or more treatments for the determined pathology.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Non-limiting and non-exhaustive examples are described with reference to the following Figures.

FIG. 1A illustrates a system for analyzing images received from an ultrasound probe according to an example.

FIG. 1B illustrates the musculoskeletal diagnosis system of FIG. 1A generating a report according to an example.

FIG. 2 illustrates a method for generating a report based on ultrasound images received from an ultrasound probe according to an example.

FIG. 3 is a block diagram illustrating example physical components of a computing device with which aspects of the disclosure may be practiced.

FIG. 4 illustrates an example diagnostic report according to an example.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Examples may be practiced as methods, systems or devices. Accordingly, examples may take the form of a hardware implementation, an entirely software implementation, or an implementation combining software and hardware aspects. The following detailed description is therefore not to be taken in a limiting sense, and the scope of the present disclosure is defined by the appended claims and their equivalents.

A musculoskeletal ultrasound probe may be used to detect a number of different musculoskeletal conditions such as tears, strains, fractures and the like. Additionally, an ultrasound probe may enable the detection of various pathologies such as arthritis, bursitis, osteoarthritis, tendinitis, etc. Ultrasound probes are typically operated by ultrasound technicians or sonographers. For example, an ultrasound technician uses the ultrasound probe to capture a number of different images of an area of interest (e.g., a knee of the patient) and subsequently provides the images to a doctor for further analysis/diagnosis. However, this process is typically inefficient.

In order to address the above, the present application describes a musculoskeletal diagnosis system that receives ultrasound and/or doppler images (collectively referred to herein as “images”) from an ultrasound probe, causes the images to be analyzed to identify different pathologies (e.g., fractures, effusions, bursitis, dislocations, arthritis, tendon injuries, blood flow, etc.) in various musculoskeletal areas (e.g., tendons, muscles, ligaments, bones, etc.) and subsequently generate a report of the diagnosis. In an example, the images may be single images or a series of images (e.g., a video).

In examples, the generated report may include selected images from the captured ultrasound images. In other examples, the images may be color-coded based, at least in part, on identified musculoskeletal areas and/or pathologies included or otherwise identified in the images. The generated report may also include various notes or other details that relate to or otherwise provide information about the selected images and/or a corresponding diagnosis.

The above may be accomplished by utilizing a musculoskeletal diagnosis system that receives various images from an ultrasound probe. When the images are received, the musculoskeletal diagnosis system causes the images to be analyzed in order to determine the musculoskeletal and/or anatomical structure in the images. The musculoskeletal diagnosis system may also identify pathologies in the images or otherwise associated with the musculoskeletal and/or anatomical structures.

Once the images have been analyzed, the musculoskeletal diagnosis system identifies images that will be returned to a computing device and/or operator associated with the ultrasound probe. The images may be identified or otherwise selected based on a number of factors including, but not limited to, different views of the musculoskeletal and/or anatomical structures, clarity/quality of the images, and identified pathologies. Portions of the images may be color-coded or otherwise have identification features that call out or otherwise identify musculoskeletal and/or anatomical structures and/or determined or identified pathologies.

In some examples, the images are included within a report that is automatically generated by the musculoskeletal diagnosis system. The report may also include information about the diagnosis and/or a recommended form of treatment (e.g., physical therapy, surgery). The report may then be provided to a computing device associated with the patient and/or a computing device associated with the operator of the ultrasound probe.

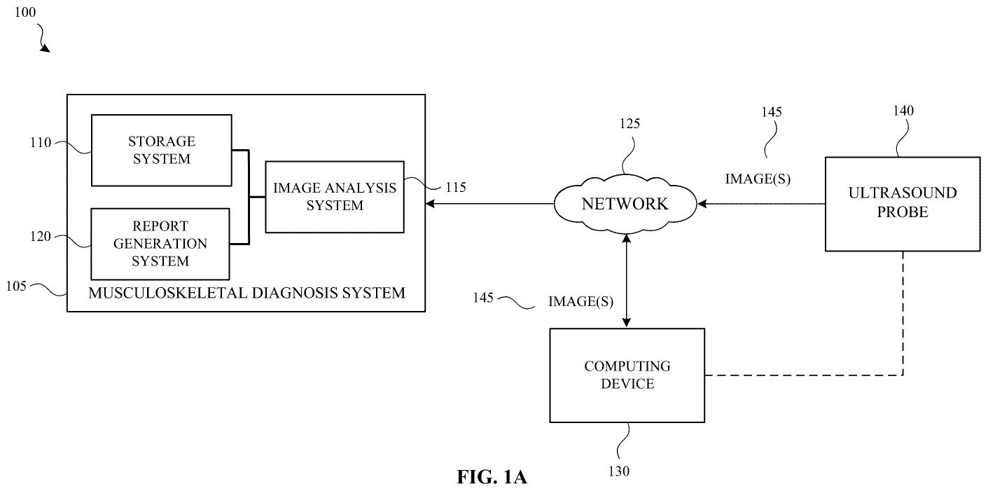

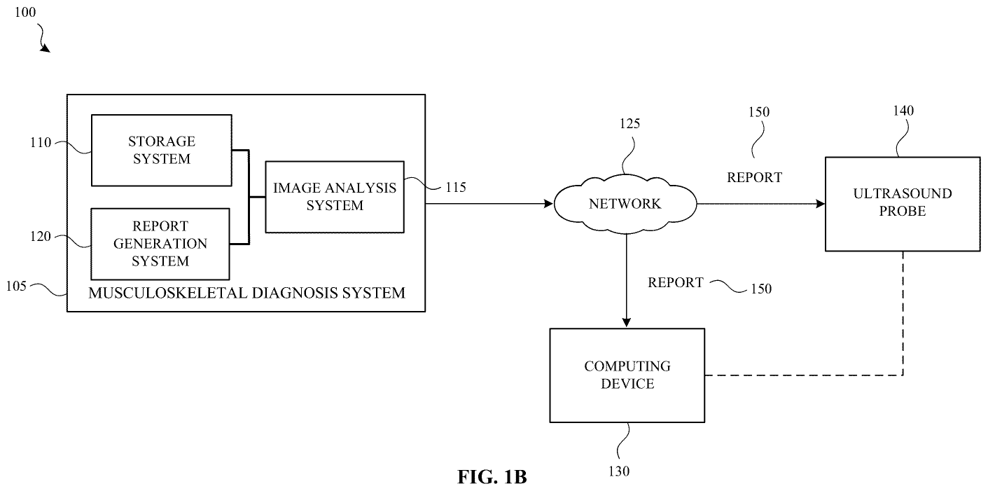

These and other examples will be shown and described in more detail with respect to FIGS. 1A-FIG. 4 below.

FIG. 1A illustrates a system 100 for analyzing images 145 received from an ultrasound probe 140 according to an example. In some examples, the system 100 may analyze images 145 captured by the ultrasound probe 140 in real-time or substantially real-time. For example, as the images 145 are captured by the ultrasound probe 140, the images 145 may be provided to a musculoskeletal diagnosis system 105 for analysis. In another example, the images 145 captured by the ultrasound probe 140 may be analyzed by the musculoskeletal diagnosis system 105 at some period of time after the images 145 are captured and/or received.

As indicated above, the system 100 includes a musculoskeletal diagnosis system 105. The musculoskeletal diagnosis system 105 may be communicatively coupled to the ultrasound probe 140 via a network 125. The musculoskeletal diagnosis system 105 may also be communicatively coupled to a computing device 130 via the network 125. Although a network 125 is specifically shown and described, the musculoskeletal diagnosis system 105 may be communicatively coupled to the ultrasound probe 140 and/or to the computing device 130 through various communication protocols including, but not limited to, Bluetooth, near-field communication, or other wireless (or wired) communication protocols.

Additionally, although the musculoskeletal diagnosis system 105 is shown as being a separate system from the computing device 130, the musculoskeletal diagnosis system 105 may be part of or otherwise integrated with the computing device 130. Likewise, the musculoskeletal diagnosis system 105 may be integrated or otherwise associated with the ultrasound probe 140. Although one computing device 130 and one ultrasound probe 140 are shown and described, the system 100 may include any number of ultrasound probes 140 and/or computing devices 130.

As shown in FIG. 1, the musculoskeletal diagnosis system 105 includes a storage system 110, an image analysis system 115, and a report generation system 120. The storage system 110 may store various images 145 that are received by the musculoskeletal diagnosis system 105. The storage system 110 may also store reports that are generated by the report generation system 120. In other examples, the storage system 110 may store instructions regarding how the images 145 are to be analyzed by the image analysis system 115.

In an example, an operator of the ultrasound probe 140 may capture various images 145 of an anatomical structure (e.g., knee, shoulder) of an individual. The images 145 are provided to the musculoskeletal diagnosis system 105 via the network 125. In examples, the images 145 are provided to musculoskeletal diagnosis system 105 in real-time or substantially real-time. Although images are specifically mentioned and described, the musculoskeletal diagnosis system 105 may receive various types of images and/or data from the ultrasound probe 140.

The images and/or other data, either alone or in combination, may be used to identify the musculoskeletal area associated with the images and/or identify a pathology associated with the musculoskeletal area. For example, an image analysis system 115 may receive the images 145, identify the musculoskeletal area contained within the image and also identify a pathology. The image analysis system 115 may also color code one or more musculoskeletal areas and/or anatomical structures contained within the image and/or color code the identified pathology.

For example, when the musculoskeletal diagnosis system 105 receives the images 145 from the ultrasound probe 140, the image analysis system 115 analyzes the images 145 by comparing the received images 145 with one or more stored images in order to identify the anatomical structure contained in the images 145 and/or identify a pathology in the images 145. For example, if the image 145 is an image of a knee, the image analysis system may determine that the patient has a meniscus tear based on comparing the image 145 to the stored images of knees with meniscus tears. In another example, the images 145 may be provided in real-time or substantially real time to sonographer that may view the images and provide a diagnosis as the images are received. In an example, the sonographer may be associated with the computing device 130 or otherwise have access to the information received by the musculoskeletal diagnosis system 105.

In another example, the image analysis system 115 may compare the images 145 to various stored images to identify the musculoskeletal area captured in the image 145 (e.g., whether the image is a knee, a tendon in the knee, a shoulder, etc.). In yet another example, additional data may be provided with the images 145. The additional data may include voice data, text data or other information that indicates the musculoskeletal area captured in the images 145. The additional data may also include information corresponding to the operator's (e.g., the operator of the ultrasound probe 140) opinion of what the pathology might be based, at least in part, on the operator's medical opinion.

When images 145 are received by the musculoskeletal diagnosis system 105, the image analysis system 115 may compare one or more patterns and/or shapes in the images 145 to one or more patterns and/or shapes in the stored images to identify the pathology and/or a musculoskeletal area on which the ultrasound probe 140 is placed. In another example, the image analysis system 115 may compare a determined or identified echogenicity within the image to an echogenicity of comparable musculoskeletal areas. In yet another example, the image analysis system 115 may compare a determined or identified echotexture of the captured musculoskeletal area within the image 145 to an echotexture of comparable musculoskeletal areas in the stored images in order to identify a pathology or other potential issue.

In another example, when various characteristics, patterns, shapes, echogenicity, echotexture and the like are identified in the image 145, the image analysis system 115 may be able to identify the pathology and/or musculoskeletal area based on stored information. In another example, the image analysis system 115 may use any number of machine learning and/or artificial intelligence techniques in order to provide a preliminary diagnosis of the pathology in the image 145 including but not limited to, object recognition, pattern matching, prediction algorithms and the like.

In yet another example and as explained above, a sonographer may analyze the images 145 and provide the diagnosis. In another example, the sonographer may analyze or otherwise review output from the image analysis system 115 and provide verification and/or corrections to the determined analysis as needed.

The image analysis system 115 may also select a subset of images that is to be included in a generated report. The subset of images are selected from the received images 145 based on one or more characteristics of each of the images. The characteristics may include how well a particular image shows an identified pathology, how well a particular image shows a particular musculoskeletal area, a clarity and/or quality of a particular image and the like.

When the image analysis system 115 has analyzed the images, selected a subset of images and/or analyzed the additional data, the information is provided to the report generation system 120. The report generation system 120 may utilize the information to generate a report. For example and referring to FIG. 1B, once the report 150 is generated, the report 150 may be provided, via the network 125, to the computing device 130 and/or the ultrasound probe 140 (or a computing device associated with the ultrasound probe 140).

In an example, the report 150 may include the subset of images that include the musculoskeletal area, a detected pathology or other potential issue that was identified by the image analysis system 115. The report 150 may also include various fields that are automatically populated based, at least in part, on the analysis of the images 145 and/or the additional information. In yet another example, the various fields may be populated by information provided by a sonographer that analyzed the images 145. In some example, the report 150 and/or the information in the report 150 may be associated with a confidence threshold. The confidence threshold may indicate an accuracy of the information (e.g., the diagnosis) contained in the report 150. As indicated above, the report may be generated and provided to the computing device 130 in real-time or substantially real-time.

As indicated above, the report 150 may include a subset of the images 145. The subset of images may be selected by the image analysis system 115 as having the highest quality when compared to the other captured images (e.g., the images included in the diagnosis are those images in which the pathology is clearly defined and/or shown). In another example, the report 150 may include one or more images (e.g., a series of images) that include optimal images (e.g., the images that provide the most detail when compared with the other captured images in the series of images). The report 150 may include or otherwise be associated with a color scheme. Each color in the color scheme may be associated with a particular pathology.

For example, the report 150 may show a meniscus tear may by a blue highlight, a blue circle or other visual indicator. In another example, the report may show bursitis using a yellow highlight, circle or other visual indicator.

In an example, a shading/opacity of the visual indicator associated with the particular pathology may be based on a confidence level associated with the information in the report. For example, an identified area of interest in images contained in the report 150 may have a first opacity if the musculoskeletal diagnosis system 105 has a first confidence level that the diagnosis is correct. In another example, an identified area of interest in the images contained in the report 150 may have a second opacity if the musculoskeletal diagnosis system 105 has a second confidence level (e.g., a higher confidence level when compared to the first confidence level) that the diagnosis is correct.

In yet another example, a shading, highlight and/or color of the visual indicator associated with the particular pathology or other detected issue may be based, at least in part, on a determined severity. For example, red visual indicator that identifies a small meniscus tear may be semi-transparent while a red visual indicator that identifies a larger meniscus tear may be more opaque.

As each report 150 is generated, the report 150 may be securely stored by the storage system 110.

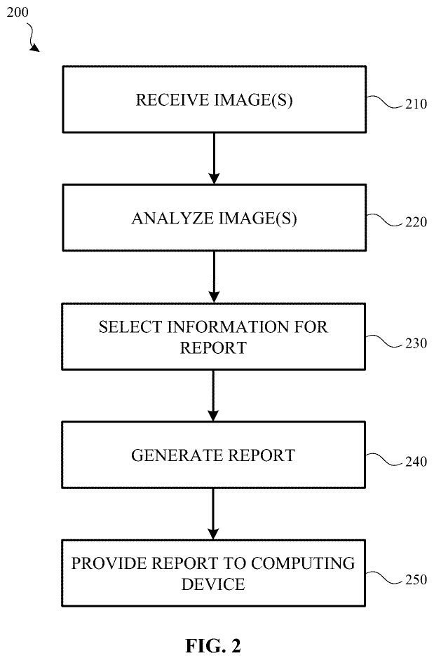

FIG. 2 illustrates a method 200 for automatically generating a report based, at least in part, on images received from an ultrasound probe according to an example. The method 200 may be used or otherwise performed by the musculoskeletal diagnosis system 105 shown and described with respect to FIG. 1A and FIG. 1B.

Method 200 begins when the musculoskeletal diagnosis system receives (210) one or more images. The images may be received from an ultrasound probe such as described above. In another example, the images may be received from a computing device associated with an ultrasound probe. In yet another example, the musculoskeletal diagnosis system resides in the cloud or is otherwise remote from an apparatus that captures ultrasound images. As such, various doctors, nurses, ultrasound technicians or other healthcare providers may send various captured images to the musculoskeletal diagnosis system via a network or other communication protocol.

In addition to receiving the images, the musculoskeletal diagnosis system may also receive additional data. The additional data may include information provided by the operator of the ultrasound probe. For example, the additional data may include a potential diagnosis, a musculoskeletal area, patient information, operation information and the like.

Once the images are received, an image analysis system of the musculoskeletal diagnosis system analyzes (220) the images. In an example, the images and/or the additional information are analyzed to identify or otherwise determine a pathology or other potential issue in the captured images. The pathology may be detected or otherwise identified by comparing various textures, patterns, etc. within the captured image to various textures, patterns, etc. of the stored images such as described above. In another example, the images and/or additional information may be analyzed to determine the musculoskeletal area captured by the images.

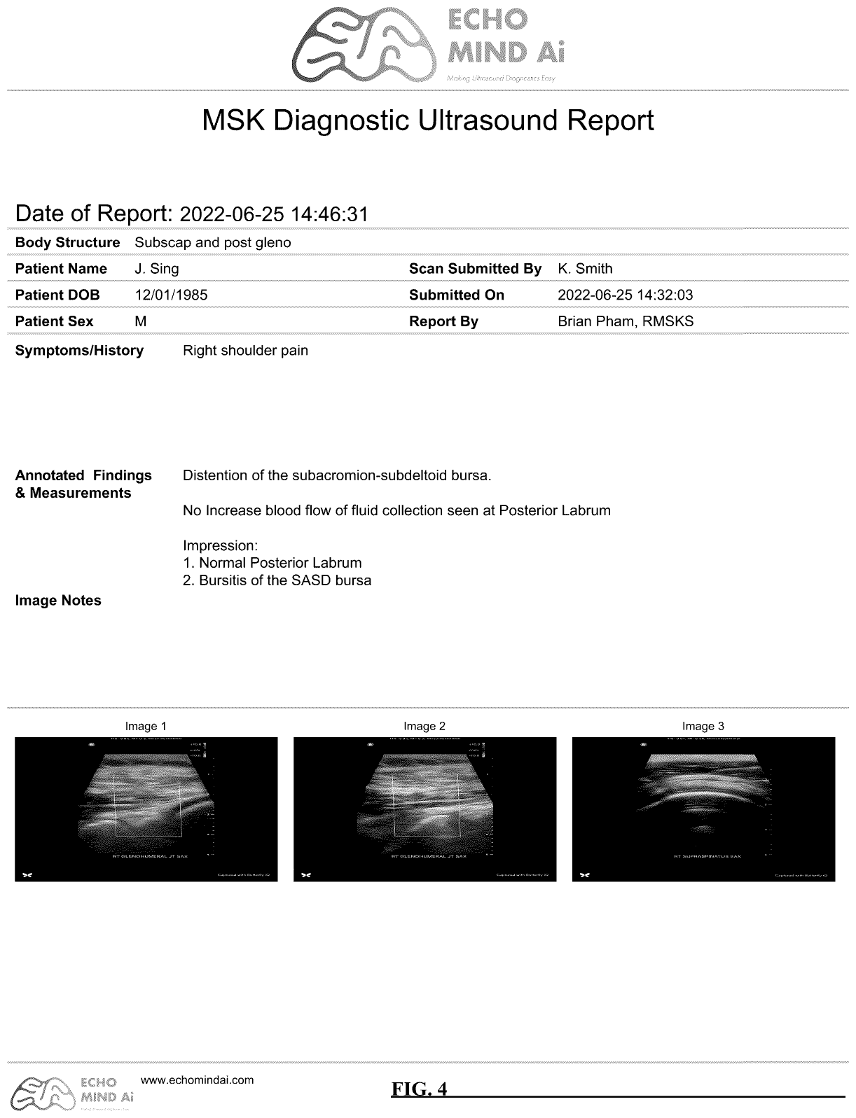

The image analysis system may also identify or otherwise select (230) a subset of images and/or automatically generate information (e.g., diagnosis information) that is to be included in a generated report. An example report is shown in FIG. 4.

In one example, the images may be selected based on various criteria such as described above. For example, a particular image or series of images may be selected based on a determination that the images best illustrate a determined diagnosis. As such, the image analysis system may identify and/or color code particular images and/or frames (sequential and/or non-sequential frames) and provide the particular images and/or frames to a report generation system.

The automatically generated information and/or the selected images are then provided to a report generation system. The report generation system automatically generates a report using the received images and information. In an example, the generated report may include a standard format. In other examples, reports may be generated in a format specified by an operator of the ultrasound probe. In another example, the format of the report may be based, at least in part, on a determine pathology and/or a determine musculoskeletal area.

Once the report is generated, the musculoskeletal diagnosis system provides (250) the generated report to a computing device.

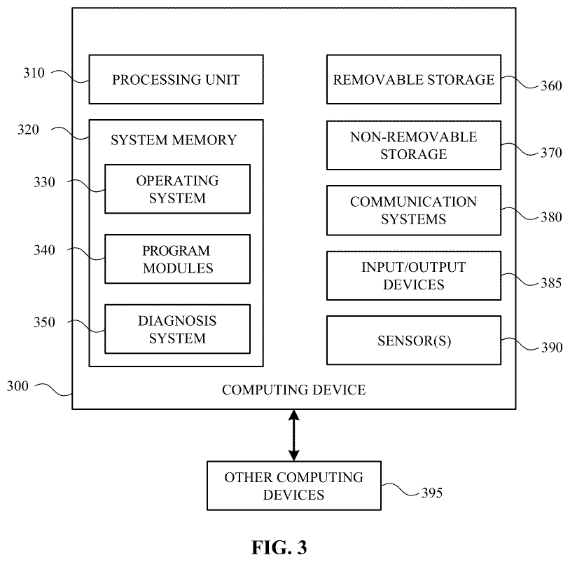

FIG. 3 and its associated descriptions provide a discussion of an example computing device that may be used with the various systems described herein. However, the illustrated computing device is an example and is not limiting as a vast number of electronic device configurations may be utilized for practicing various aspects of the disclosure.

FIG. 3 is a block diagram illustrating physical components (e.g., hardware) of a computing device 300 with which aspects of the disclosure may be practiced. The computing device 300 may be integrated or otherwise associated with any of the various systems described above with respect to FIG. 1A and FIG. 1B. For example, the computing device 300 may be integrated or otherwise associated with an ultrasound probe, the musculoskeletal diagnosis system 105, the computing device 130, the image analysis system 115, the instruction system 120 and/or the storage system 110.

In a basic configuration, the computing device 300 may include at least one processing unit 310 and a system memory 320. Depending on the configuration and type of computing device, the system memory 320 may comprise, but is not limited to, volatile storage (e.g., random access memory), non-volatile storage (e.g., read-only memory), flash memory, or any combination of such memories. The system memory 320 may include an operating system 330 and one or more program modules 340 or components suitable for performing the various operations described above. The operating system 330 may be suitable for controlling the operation of the computing device 300. The system memory 320 may include a diagnosis system 350.

The computing device 300 may have additional features or functionality. For example, the computing device 300 may also include additional data storage devices (removable and/or non-removable) such as, for example, magnetic disks, optical disks, or tape. Such additional storage is illustrated in FIG. 3 by a removable storage device 360 and a non-removable storage device 370.

As stated above, a number of program modules 340 and data files may be stored in the system memory 320. While executing on the processing unit 310, the program modules 340 may perform the various processes including, but not limited to, the aspects, as described herein.

Furthermore, examples of the disclosure may be practiced in an electrical circuit comprising discrete electronic elements, packaged or integrated electronic chips containing logic gates, a circuit utilizing a microprocessor, or on a single chip containing electronic elements or microprocessors. For example, examples of the disclosure may be practiced via a system-on-a-chip (SOC) where each or many of the components illustrated in FIG. 3 may be integrated onto a single integrated circuit. Such an SOC device may include one or more processing units, graphics units, communications units, system virtualization units and various application functionality all of which are integrated (or “burned”) onto the chip substrate as a single integrated circuit.

When operating via an SOC, the functionality, described herein, with respect to the capability of client to switch protocols may be operated via application-specific logic integrated with other components of the computing device 300 on the single integrated circuit (chip). Examples of the disclosure may also be practiced using other technologies capable of performing logical operations such as, for example, AND, OR, and NOT, including but not limited to mechanical, optical, fluidic, and quantum technologies. In addition, examples of the disclosure may be practiced within a general-purpose computer or in any other circuits or systems.

The computing device 300 may also have one or more input/output device(s) 385. These include, but are not limited to, a keyboard, a trackpad, a mouse, a pen, a sound or voice input device, a touch, force and/or swipe input device, a display, speakers, a printer, etc. The aforementioned devices are examples and others may be used. The computing device 300 may include one or more communication systems 380 that allow or otherwise enable the computing device 300 to communicate with remote computing devices 395. Examples of suitable communication connections include, but are not limited to, radio frequency (RF) transmitter, receiver, and/or transceiver circuitry; universal serial bus (USB), parallel, and/or serial ports.

The computing device may include one or more sensors 390. The sensors may include location sensors, accelerometers, position sensors the like.

The term computer-readable media as used herein may include computer storage media. Computer storage media may include volatile and nonvolatile, removable and non-removable media implemented in any method or technology for storage of information, such as computer readable instructions, data structures, or program modules.

The system memory 320, the removable storage device 360, and the non-removable storage device 370 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 300. Any such computer storage media may be part of the computing device 300. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. In addition, each of the operations described above may be executed in any order. For example, one operation may be performed before another operation. Additionally, one or more of the disclosed operations may be performed simultaneously or substantially simultaneously.

Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

1. A method, comprising:
receiving an image from an ultrasound probe;
causing the image to be analyzed;
identifying a pathology in the image;
automatically generating a report; and
providing the report to a computing device.
